(19) United States
(12) Patent Application Publication
(10) Pub. No.: US 2018/0020992 A1
(43) Pub. Date: Jan. 25, 2018

(54) SYSTEMS AND METHODS FOR MEDICAL VISUALIZATION

(71) Applicant: Dimensions and Shapes, LLC, Tampa, FL (US)

(72) Inventor: Yanhui GUO, Miami, FL (US)

(73) Assignee: Dimensions and Shapes, LLC, Tampa, FL (US)

(21) Appl. No.: 15/549,851

(22) PCT Filed: Feb. 15, 2016
(86) PCT No.: PCT/US2016/017963

§ 371 Date: Aug. 9, 2017

Related U.S. Application Data
(60) Provisional application No. 62/116,824, filed on Feb. 16, 2015.

(51) Int. Cl. A61B 5/00; A61B 6/03; A61B 8/00; A61B 6/00; G06T 7/00; A61B 5/06; A61B 6/12; G02B 27/01; G06T 19/00; G06T 7/11; A61B 5/055; A61B 8/08

(52) U.S. Cl. CPC A61B 5/7425; A61B 5/055; A61B 6/032; A61B 8/466; A61B 6/466; A61B 8/483; A61B 5/061; A61B 6/12; A61B 8/0841; A61B 5/0015; G02B 27/0172; G06T 19/006; G06T 7/11; G06T 7/0012; G02B 2027/0134; G02B 2027/014; G06T 2210/41; G06T 2207/10081; G06T 2207/10088; G06T 2207/30096

(57) ABSTRACT

A visualization system includes an imaging device, a processing circuit, and a display device. The imaging device is configured to acquire image data relating to an object. The processing circuit is configured to generate a three-dimensional object image of the object based on the image data and generate a two-dimensional object image of the object by projecting a cross-section of the three-dimensional object image onto a plane. The display device is configured to display the three-dimensional object image of the object and display the two-dimensional object image of the object on the plane.

### CROSS-REFERENCE TO RELATED PATENT APPLICATIONS

This application claims the benefit of U.S. Provisional Patent Application No. 62/116,824, titled “Systems and Methods for Medical Visualization,” filed Feb. 16, 2015, which is incorporated herein by reference in its entirety.

### TECHNICAL FIELD

The present disclosure relates to systems and methods for medical visualization and more particularly to systems and methods for medical visualization based on augmented reality.

### BACKGROUND

Medical imaging systems, such as ultrasound-based imaging systems, computed tomography (CT) based imaging systems, and Magnetic Resonance Imaging (MRI) based imaging systems, are used to investigate the anatomy of the body for diagnostic purposes. Traditional medical imaging systems may visualize an internal body structure by forming two-dimensional (2D) images of the body structure in different directions, and displaying them on respective 2D views. For example, as shown in FIG. 1, traditional medical imaging systems may display an internal body structure by forming an anteroposterior image 11 and an inferosuperior image 21, and displaying them on respective 2D views—an anteroposterior view 10 and an inferosuperior view 20. With these 2D views, however, most health care professionals need more information with real clinical relevance. Alternatively, traditional medical imaging systems may generate three-dimensional (3D) images by acquiring a number of adjacent 2D images and displaying the 3D images using 3D visualization models. For example, as shown in FIG. 2, a 3D box image 31 may be created by filling it with a plurality of 2D image slices (e.g., more than 300, etc.). However, without further visual analysis, it is impossible for the human eyes to see through the filled image slices, and therefore such 3D visualization does not provide healthcare professionals with clinically useful information.

Meanwhile, augmented reality (AR) based or virtual reality (VR) based visualization techniques are used in many applications including military, industrial, and medical applications. More particularly, AR may provide healthcare professionals with a variety of information by combining multiple visualization sources, like images, and videos. AR may also be used to enhance visualization of an internal body structure by combining computer graphics with images of the internal body.

While 3D visualization techniques are used to visualize AR views in a 3D space for a medical test or surgery, traditional visualization techniques do not effectively guide a medical tool (e.g., a biopsy needle, a medical scissor, etc.) so as to help a health professional orient themselves in the 3D space. Therefore, there is a need for visualizing AR views in real time to accurately guide the medical tool to a target tissue area so as to more effectively aid the professional to perform a medical test or surgery.

### SUMMARY

One embodiment relates to a visualization system. The visualization system includes an imaging device, a processing circuit, and a display device. The imaging device is configured to acquire image data relating to an object. The processing circuit is configured to generate a three-dimensional object image of the object based on the image data and generate a two-dimensional object image of the object by projecting a cross-section of the three-dimensional object image onto a plane. The display device is configured to display the three-dimensional object image of the object and display the two-dimensional object image of the object on the plane.

Another embodiment relates to a method for visualizing objects using an augmented reality based visualization system. The method includes acquiring, by an imaging device, image data relating to an object; generating, by a processing circuit, a three-dimensional object image of the object based on the image data; projecting, by the processing circuit, a two-dimensional object image of the object onto a plane; and displaying, by a display device, at least one of the three-dimensional object image and the two-dimensional object image.

Still another embodiment relates to a visualization system. The visualization system includes a processing circuit communicably and operatively coupled to an imaging device and a display device. The processing circuit is configured to receive image data from the imaging device regarding an object, generate a three-dimensional image of the object based on the image data, set a first plane and a second plane intersecting the first plane, generate a first two-dimensional image of the object by projecting a first cross-section of the three-dimensional image of the object onto the first plane, generate a second two-dimensional image of the object by projecting a second cross-section of the three-dimensional image onto the second plane, and provide a command to the display device to display the three-dimensional image of the object between the first plane and the second plane, the first two-dimensional image of the object on the first plane, and the second two-dimensional image of the object on the second plane.

The foregoing summary is illustrative only and is not intended to be in any way limiting. In addition to the illustrative aspects, embodiments, and features described above, further aspects, embodiments, and features will become apparent by reference to the drawings and the following detailed description. Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 shows exemplary 2D views of an object generated by a traditional visualization system.

FIG. 2 is an exemplary 3D view of an object generated by a traditional visualization system.

FIG. 3 is a schematic block diagram of a visualization system, according to an exemplary embodiment.

FIGS. 4A and 4B are views of a visualization system, according to an exemplary embodiment.

FIGS. 5A and 5B are exemplary 3D views of an object generated by a visualization system, according to an exemplary embodiment.

FIGS. 6A and 6B are exemplary 3D views of an object generated by a visualization system, according to an exemplary embodiment.

FIGS. 7A and 7B are exemplary 3D views of an object and a tool generated by a visualization system, according to an exemplary embodiment.

FIG. 8 is a flow diagram of a method for providing a display of an object and/or a tool by a visualization system, according to an exemplary embodiment.

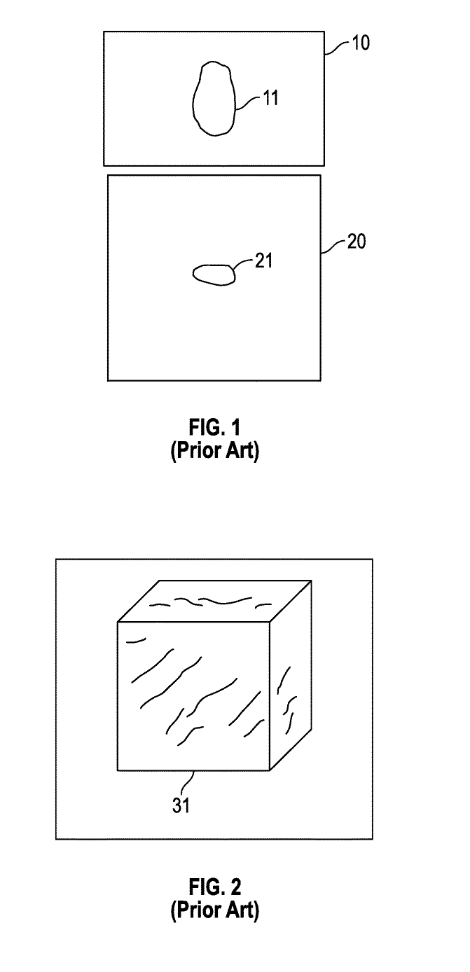

### DETAILED DESCRIPTION

In the following detailed description, reference is made to the accompanying drawings, which form a part thereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Referring to the Figures generally, various embodiments disclosed herein relate to a visualization system capable of providing a 3D image of an object (e.g., an internal body part, a lesion, etc.) along with its projected images on 2D planes. The visualization system allows a user (e.g., a health professional, surgeon, veterinarian, etc.) to easily orient themselves in 3D space. The visualization system is also configured to display a 3D image of a tool (e.g., needle, scissors, etc.) along with its projected images on the same 2D planes so that the tool may be accurately guided to a target tissue area, thereby effectively aiding the user to perform a medical test, surgery, and/or procedure.

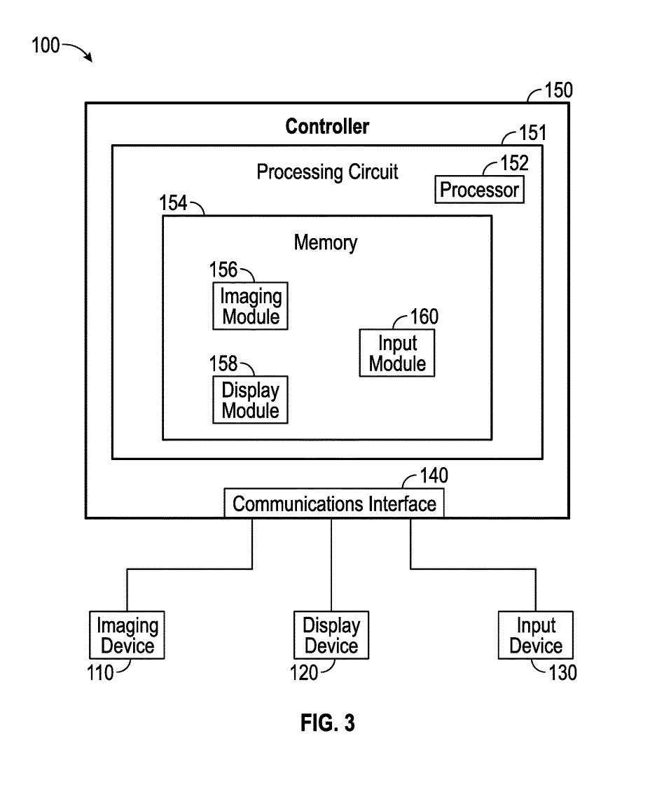

According to the exemplary embodiment shown in FIG. 3, a visualization system 100 includes an imaging device 110, a display device 120, an input device 130, and a controller 150. According to an exemplary embodiment, the visualization system 100 is an augmented reality based visualization system. The imaging device 110 may be configured to monitor a region of interest (e.g., including a tumor, lesion, organ, etc.; a tool such as a needle, a scissors, etc.; etc.) and gather data (e.g., imaging data, etc.) regarding the region of interest and/or other objects (e.g., a tool such as a needle or scissors, etc.). The imaging device 110 may include, but is not limited to, an ultrasound device, a computed tomography (CT) device, or a magnetic resonance imaging (MRI) device, among other alternatives. In some embodiments, the imaging device 110 includes a transducer (e.g., an ultrasound transducer, etc.) configured to acquire the imagine data.

The display device 120 may be configured to display at least one of a three-dimensional (3D) reconstruction of the region of interest, one or more two-dimensional (2D) projections of the 3D reconstruction of the region of interest, a 3D reconstruction of a tool in or near the region of interest, and one or more 2D projections of the reconstruction of the tool to a user based on the imaging data acquired by the imaging device 110. The display device 120 may include a light emitting diode (LED) display, a liquid-crystal display (LCD), a plasma display, a cathode ray tube (CRT), a projector, a portable device (e.g., a smartphone, tablet, laptop, augmented reality glasses, etc.), and/or any other type of display device. The display device 120 may additionally or alternatively include a head-mount device capable of displaying AR and/or VR views. The display device 120 may include a tilt sensor that can detect the tilting of the display device 120 so that the displayed view may be adjusted according to the detected tilting.

The input device 130 may allow a user of the visualization system 100 to communicate with the visualization system 100 and the controller 150 to adjust the display provided on the display device 120 or select certain features provided by the visualization system 100. For example, the input device 130 may include, but is not limited, an interactive display, a touchscreen device, one or more buttons and switches, voice command receivers, a keyboard, a mouse, a track pad, etc. The input device 130 may be configured to allow the user of the visualization system 100 to maneuver the 3D reconstructions and/or 2D projections including rotation, inversion, translation, magnification, selection of a specific portion of the 3D reconstruction, and the like. The input device 130 may further allow a user to customize the 3D reconstruction such as change a color and an opaqueness/transparency, among other possibilities.

The controller 150 may include a communications interface 140. The communications interface 140 may include wired or wireless interfaces (e.g., jacks, antennas, transmitters, receivers, transceivers, wire terminals, etc.) for conducting data communications with various systems, devices, or networks. For example, the communications interface 140 may include an Ethernet card and port for sending and receiving data via an Ethernet-based communications network and/or a WiFi transceiver for communicating via a wireless communications network. The communications interface 140 may be configured to communicate via local area networks or wide area networks (e.g., the Internet, a building WAN, etc.) and may use a variety of communications protocols (e.g., BACnet, IP, LON, Bluetooth, ZigBee, radio, cellular, etc.).

The communications interface 140 may be a network interface configured to facilitate electronic data communications between the controller 150 and various external systems or devices of the visualization system 100 (e.g., the input device 130, the display device 120, the imaging device 110, etc.). By way of example, the controller 150 may receive one or more inputs from the input device 130. By way of another example, the controller 150 may receive data (e.g., imaging data, etc.) from the imaging device 110 regarding one or more regions of interest or objects (e.g., tumors, lesions, organs, tools, etc.).

As shown in FIG. 3, the controller 150 includes a processing circuit 151 including a processor 152 and a memory 154. The processor 152 may be implemented as a general-purpose processor, an application specific integrated circuit (ASIC), one or more field programmable gate arrays (FPGAs), a digital signal processor (DSP), a group of processing components, or other suitable electronic processing components. The one or more memory devices 154 (e.g., RAM, ROM, Flash Memory, hard disk storage, etc.) may store data and/or computer code for facilitating the various processes described herein. Thus, the one or more memory devices 154 may be communicably connected to the processor 152 and provide computer code or instructions to the processor 152 for executing the processes described in regard to the controller 150 herein. Moreover, the one or more memory devices 154 may be or include tangible, non-transient volatile memory or non-volatile memory. Accordingly, the one or more memory devices 154 may include database components, object code components, script components, or any other type of information structure for supporting the various activities and information structures described herein.

The memory 154 may include various modules for completing the activities described herein. More particularly, the memory includes modules configured to generate a 3D image of a region of interest to aid a user in a designated task (e.g., remove a tumor with greater precision, inspect a region of interest to obtain information, etc.). While various modules with particular functionality may be include, it should be understood that the controller 150 and memory 154 may include any number of modules for completing the functions described herein. For example, the activities of multiple modules may be combined as a single module, additional modules with additional functionality may be included, etc. Further, it should be understood that the controller 150 may further control other functions of the visualization system 100 beyond the scope of the present disclosure.

As shown in FIG. 3, the controller 150 includes an imaging module 156, a display module 158, and an input module 160. The imaging module 156 may be operatively and/or communicably coupled to the imaging device 110 and configured to receive and store the imaging data acquired by the imaging device 110. The imaging module 156 may interpret the imaging data to generate (e.g., construct, etc.) a 3D image of the region of interest which may include an object such as a tumor, an organ, and/or a lesion. Further, the imaging module 156 may be configured to generate 2D projections of the 3D image. In one embodiment, a first 2D projection is constructed from a cross-section of the 3D image along a first plane (e.g., an x-y plane, etc.) through the center of the 3D image along a first axis (e.g., a z-axis, etc.). In some embodiments, a second 2D projection is constructed from a cross-section of the 3D image along a second plane (e.g., an x-z plane, etc.) through the center of the 3D image along a second axis (e.g., a y-axis, etc.). In other embodiments, a third 2D projection is constructed from a cross-section of the 3D image along a third plane (e.g., a y-z plane, etc.) through the center of the 3D image along a third axis (etc. an x-axis, etc.).

According to an exemplary embodiment, the user may select which of the projections are generated (e.g., the first, second, third, etc. protection) and/or where along the respective axis the cross-section of the object is taken to generate a customized 2D cross-sectional projection of the object. In an alternative embodiment, the user is able to select any plane in a three-dimensional space at which the cross-section is taken for the 2D projection (e.g., an angled plane, a plane at an angle to at least one of the x- axis, y-axis, and z-axis, etc.). The imaging module 156 may further generate a 3D representation of a tool that the imaging data indicates is in or near the region of interest. The imaging module 156 may generate 2D projections of the tool, such that the 2D projections of the tool aid in understanding the spatial relationship between the tool and the region of interest (e.g., tumor, organ, lesion, etc.). The imaging module 156 may further predict the path of travel of the tool and communicate the prediction to the display module 158 to be displayed to the user to aid in the insertion of the tool.

The display module 158 may be communicably coupled to the display device 120 and configured to display the images (e.g., 2D projections of the region of interest and/or tool, 3D reconstruction of the region of interest and/or tool, etc.) generated by the imaging module 156 to the user on the display device 120. In one embodiment, the display module 158 displays at least one of a 3D reconstruction of the region of interest, one or more 2D projections of the 3D reconstruction of the region of interest, a 3D reconstruction of the tool, one or more 2D projections of the 3D reconstruction of the tool, and a predicted path of travel of the tool on a single screen. Additionally or alternatively, the display module 158 may segment the display and show each 2D view in a designated window. The user may add more windows to see more views simultaneously or remove the additional windows to show everything in one cumulative display. With the various displayed images, the user of the visualization system 100 may be able to spatially orient themselves with regard to the region of interest such that the procedure or task to be performed is able to be done with a greater precision and understanding of the region of interest and the object (e.g., a tumor, lesion, organ, etc.). Also, the 3D reconstruction of the tool allows the user to identify if the object in the region of interest is below, above, in front of, behind, to the left of, and/or to the right of the tool to facilitate precise actions. The display module 158 may also display the predicted path of the tool such that the user may see the trajectory at which the tool is heading so he/she may make real-time decisions to follow the current path or manipulate the tool to follow an altered path to better perform the required task. In a medical setting, this may allow for more of a tumor or lesion to be removed while minimally compromising the surrounding healthy tissue, organs, etc.

The input module 160 may be communicably and/or operatively coupled to the input device 130 and configured to receive one or more inputs from a user of the visualization system 100. The input module 160 may also be communicably and/or operatively coupled to the display module 158 such that the user of the visualization system 100 may use the input device 130 to vary the display presented to them. The display module 158 may further provide a variety of command features to the user such as selectable buttons or drop down menus to select various options and features. In one embodiment, the display module 158 may provide the user of the visualization system 100 with a graphical user interface (GUI) on a screen of the display device 120. Using the input device 130, the user may select from the variety of displayed buttons or drop down menus provided by the display module 158.

The options provided by the display module 158 may include a color feature, a transparency feature, a spatial orientation feature, and the like. The color feature may provide the user with the ability to change the color of various objects or features on the display. For example, the user may want to differentiate between the object within the region of interest and the tool, such that the 3D reconstruction of the object is colored a first color and the 3D reconstruction of the tool is colored a different second color. As another example, the user may be able to select a color for the 2D projections that differs from the color of the 3D reconstructions. The transparency feature may facilitate changing the images shown on the display from 0% transparent (e.g., solid, not see-through, opaque, etc.) to 100% transparent (e.g., invisible, etc.). This may be applied to a whole object or portions of an object. For example, the displayed region of interest may include both a tumor and an organ. The transparency feature may provide the user with the option of viewing the tumor or the organ individually. The spatial orientation feature may provide the user with the option of spatially altering the presentation of the 3D reconstructions and/or the 2D projections. For example, the user may be able to translate the 3D image (e.g., left, right, front, back, etc.), adjust magnification (e.g., zoom in, zoom out, etc.), rotate the display (e.g., spherical rotation, multi-axis rotation, rotation in any direction, etc.), crop the image (e.g., select certain portions to focus on, etc.), and the like. As the 3D reconstruction is spatially reoriented, the 2D projections related to the 3D reconstruction may update automatically responsive to the adjusted position of the 3D reconstruction presented by the display device 120.

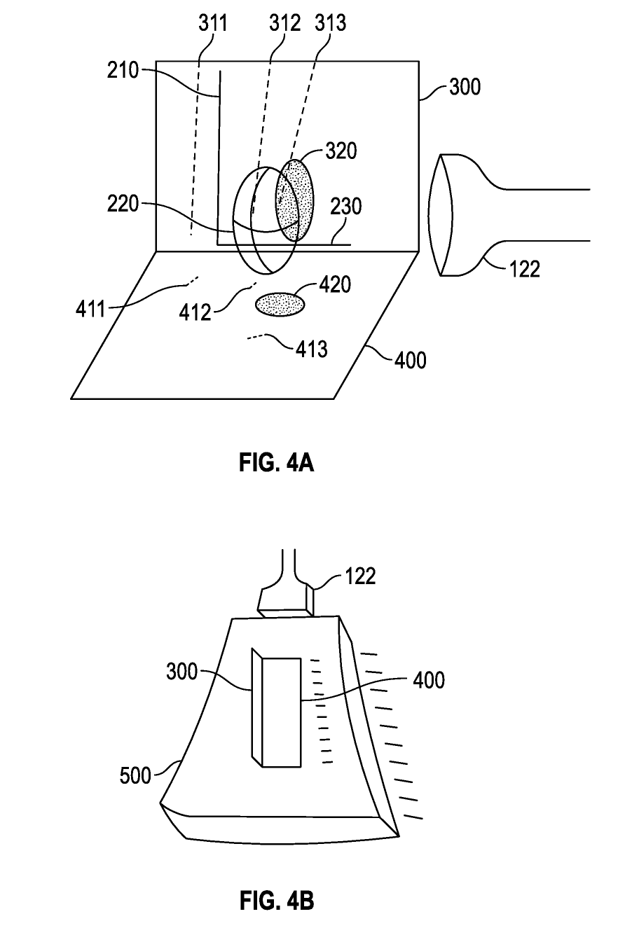

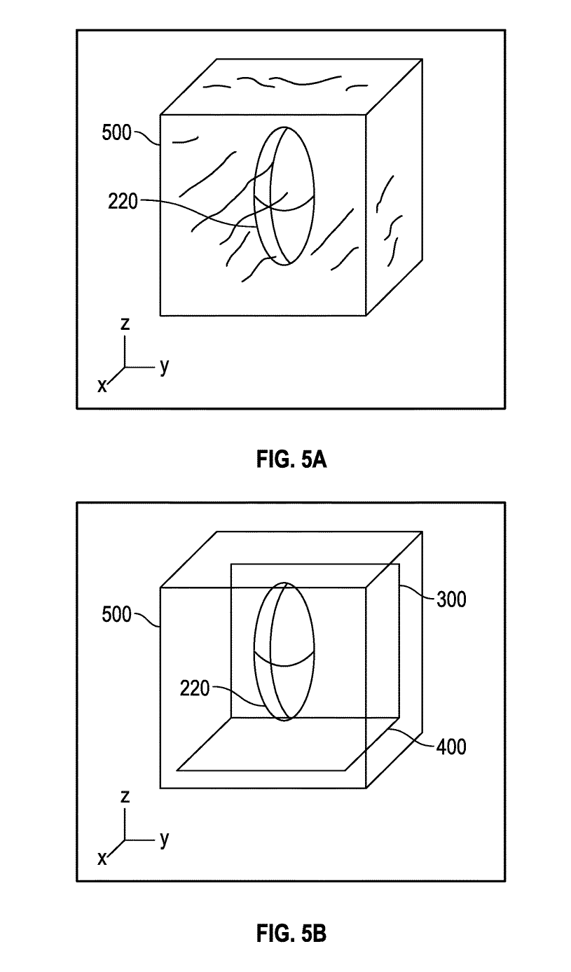

Referring now to FIGS. 4A and 4B, views of the visualization system 100 are shown, according to an exemplary embodiment. As shown in FIGS. 4A and 4B, the visualization system 100 may construct a 3D object image 220 representing an object or region of interest internal to a surface (e.g., a body part, an organ, a lesion, etc.) in a 3D region 500 using a transducer 122 of the imaging device 110. The visualization system 100 may display the 3D object image 220 between a first plane 300 and a second plane 400 in the 3D region 500 so that a user (e.g., a health professional, a surgeon, etc.) may better understand the 3D region 500 and a spatial relationship between the 3D object image 220 and the 3D region 500. The visualization system 100 may further allow the user to manipulate the 3D object image 220 in the 3D region 500 based on the spatial relationship. The user may manipulate the 3D object image 220 through various commands facilitating the rotation, magnification, translation, and the like of the 3D object image 220 on the display device 120. The 3D region 500 may be a rectangular parallelepiped (see FIG. 4B) or a cube (see FIG. 5B). 2D planes may be defined on or within the 3D region 500 (see FIGS. 4B and 5B). For example, the 3D object image 220 of the object (e.g., tumor, etc.) may be generated and visualized. The 3D object image 220 may also be accompanied by projections of the 3D object image 220, shown as a first projected object image 320 on the first plane 300 and a second projected object image 420 on the second plane 400. The 3D object image 220, as well as the first projected object image 320 and the second projected object image 420 may be displayed to the user on the display device 120. In some embodiments, additional projected object images are displayed on additional planes (e.g., a third projected object image on a third plane, etc.). In one embodiment, the images are displayed on a head-mount display device (e.g., AR glasses, AR device, etc.) so that the user may easily flip the 3D object image 220 up/down and left/right while wearing the head-mount display device. The positions of the first plane 300 and the second plane 400 and the first projected object image 320 and the second projected object image 420 thereon may be calculated in real time based on imaging data acquired by the imaging device 110 (e.g., the ultrasound data acquisition device, etc.). Alternatively, the imaging device 110 may be the CT data acquisition device or the Mill data acquisition device. In this manner, for example, the visualization system 100 may visualize, in real time, the first projected object image 320 and the second projected object image 420 along with the constructed 3D object image 220 so as to provide user with a spatial relationship regarding the images and the 3D region 500.

Referring still to FIG. 4A, the visualization system 100 may construct a 3D tool image 210 of a tool (e.g., a needle a scissor, a clip, etc.). The transducer 122 may acquire imaging data from an imaging source, e.g., ultrasound image data. With the imaging data, the visualization system 100 may generate the 3D tool image 210 of the tool within the 3D region 500. The visualization system 100 may also generate or construct projections of the 3D tool image 210 of the tool onto the first plane 300 and/or the second plane 400, respectively. The projected images are represented like shadows of the tool cast on the first plane 300 and the second plane 400 so that the user may better understand the spatial relationship between the 3D object image 220 of the object and the 3D tool image 210 of the tool. In some embodiments, additional projected tool images are displayed on additional planes (e.g., a third projected tool image on a third plane, etc.). With a better understanding of the spatial relationship between the object and the tool, the user may manipulate the tool with better knowledge, understanding, and precision. For example, FIG. 4A shows three exemplary shadows (projected 2D images of the tool) 311, 312 and 313 cast on the first plane 300 according to different positions of the tool as it was introduced to the 3D region 500, and corresponding shadows 411, 412 and 413 cast on the second plane 400.

The positions of the first plane 300 and the second plane 400 and the projected 2D images of the tool may be generated in real time based on image data acquired by the transducer 122 of the imaging device 110 (e.g., the ultrasound data acquisition device, etc.). For example, clips or tags may be attached to the tool so that the position and images of the tool may be generated from the data acquired by the imaging device 110. The projected images (e.g., of the object, internal body part, lesion, tool, etc.) may be edited with different colors. For example, the first projected object image 320 and/or the second projected object image 420 may be different in color from those of the tool projections 311-313 and/or 411-413. The color of the projected images of the tool may be changed to a different color (e.g., red, etc.) once it goes into the surface (e.g., skin, etc.). Alternatively, the lower end position of the tool may be marked with an indicator, shown as line 230, so that the user may easily recognize how far the tool reaches in the surface relative to the object. In this manner, for example, if 3D images of a tumor and a needle are visualized, as the surgeon pushes the needle into the skin, he/she may be able to recognize from the shadow 412 on the second plane 400 that the needle is behind the tumor. As the surgeon pushes the needle further, he/she may be able to see that the shadow of the needle on the first plane 300 reaches the lower edge of the first projected object image 320, and recognize that because the needle reaches the lower edge of the tumor, he/she does not need to push the needle in further.

The visualization system 100 may additionally or alternatively graphically construct/render a 3D image of a tool (e.g., a scissor, a needle, etc.) based on a graphical model (e.g., stored in the memory 154, etc.) and then visualize the 3D tool image between the first plane 300 and the second plane 400 in the 3D region 500. The visualization system 100 may also generate a projection of the 3D tool image onto the first plane 300 and the second plane 400, respectively, and then visualize the projected images on the first plane 300 and the second plane 400 like shadows of the tool cast on the respective 2D planes. For example, by visualizing the constructed 3D image of a scissor and its shadows cast on the 2D planes along with the images of a tumor, the surgeon may simulate the scissor in the 3D region to know how to proceed and remove the object (e.g., tumor, etc.).

The visualization system 100 displays the 3D images and 2D projected images thereof on the display device 120. Alternatively, the images may be displayed on portable device such as a smartphone, tablet, laptop, or AR glasses. The portable device may visualize the images in the same manner as the display device 120. The only difference is that on the smartphone, the user may tilt the portable device such that the position of the images changes with the tilt angle so the portable device may show views of the tumor and needle, for example, from different angles corresponding to the tilting. For this purpose, the display device 120 including a tilt sensor may be used.

FIGS. 5A and 5B are exemplary 3D views of an object (e.g., a tumor, etc.) generated by the visualization system 100 according to an exemplary embodiment. The controller 150 may detect the regions of interests based on preoperative analysis, and reconstruct the 3D region 500 based on the regions of interests in a real-time fashion for an improved visualization, thereby improving the understanding of the spatial relationship between the displayed objects and the subsequent treatment management of the pathologies in question.

Referring to FIG. 5B, the controller 150 may generate the 3D object image 220 of the tumor from the imaging data acquired by the imaging device 110 and set the first plane 300 and the second plane 400 in real time based on the imaging data acquired by the imaging device 110. The display device 120 may display the 3D region 500 in which the 3D object image 220 of the object is located between the first plane 300 and the second plane 400. The 3D region 500 may be a rectangular parallelepiped (see FIG. 4B) or a cube (see FIGS. 5A and 5B).

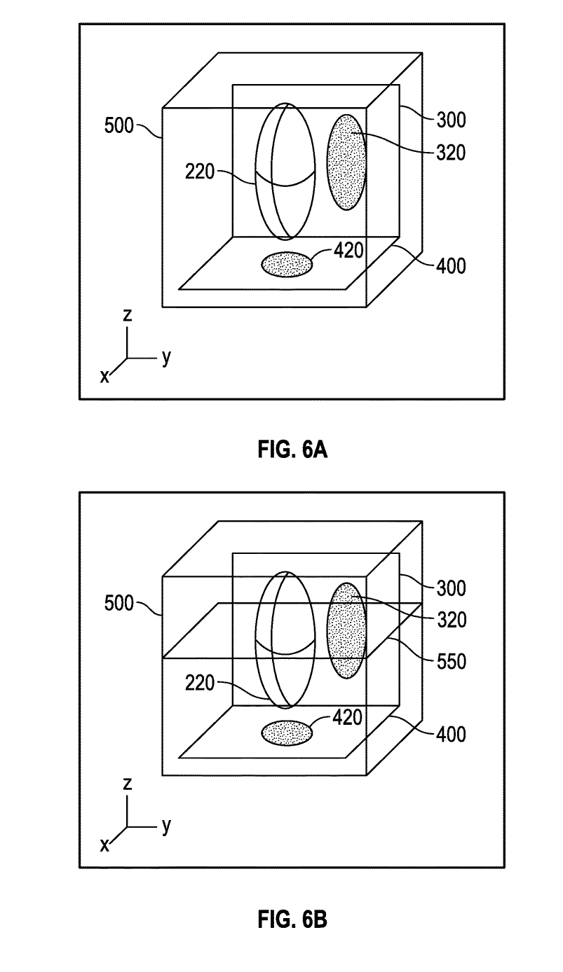

FIGS. 6A and 6B are exemplary 3D views of the object generated by the visualization system 100 according to an exemplary embodiment. The visualization system 100 generates a 3D view of the object as the 3D object image 220 and 2D projections of the 3D view including the first projected object image 320 and the second projected object image 420 on the first plane 300 and the second plane 400, respectively, such that the different 3D features of the pathologies may be evaluated. A user (e.g., a health professional, a surgeon, etc.) may use all these views to assess response to treatment, growth patterns, and other clinically important features of the object.

More particularly, referring to FIG. 6A, the controller 150 may reconstruct a 3D object image 220 representing the object in the 3D region 500 from an imaging source, e.g., image data acquired by the imaging device 110. The display device 120 then displays the reconstructed 3D object image 220 between the first plane 300 and the second plane 400 in the 3D region 500 so that a health professional, e.g. a surgeon, may better understand the 3D region and a spatial relationship between the 3D object image 220 and the 3D region 500 and manipulate the 3D object image 220 in the 3D region 500 based on the spatial relationship.

Referring to FIG. 6B, the first projected object image 320 and the second projected object image 420 may include 2D images, e.g., obtained from a cross-section of the 3D object image 220 with a plane 550 so as to confirm to the user that the visualization is accurate. Compared with FIG. 6A, the display device 120 may display the cross-sectional plane 550 in addition to the 3D region 500, the 3D object image 220, the first projected object image 320 and the second projected object image 420, and the first plane 300 and the second plane 400. The controller 150 may generate the second projected object image 420 in real time based on the image data acquired by the imaging device 110 (e.g., the ultrasound data acquisition device) that represents the data obtained from the cross-sectional plane 550. Alternatively, the first projected object image 320 and the second projected object image 420 may be generated based on image data acquired by the CT data acquisition device or the MRI data acquisition device.

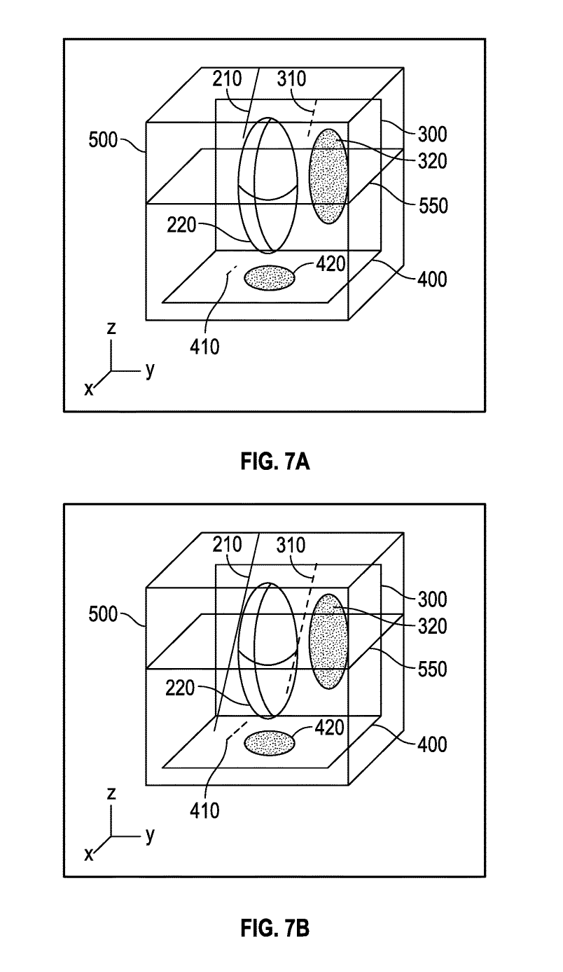

FIGS. 7A and 7B are exemplary 3D views of the object and a tool (e.g., a needle, etc.) generated by the visualization system 100 according to an exemplary embodiment. The introduction of the tool in the visualization may provide a more accurate position and orientation within the 3D space in a real-time fashion, thereby leading to an improved surgical guidance. After the correct positioning of one or more needles, the surgeon may “scoop out” the suspicious area conserving the maximum amount of healthy tissue.

More particularly, referring to FIG. 7A, the controller 150 may generate the 3D tool image 210 of the tool from an imaging source, e.g., image data acquired by the imaging device 110. The display device 120 then displays the reconstructed 3D tool image 210 of the tool between the first plane 300 and the second plane 400 in the 3D region 500. The controller 150 may also generate or reconstruct one or more projections of the 3D tool image 210 onto the first plane 300 and the second plane 400, respectively, and then display the first projected tool image 310 and the second projected tool image 410 of the tool on the first plane 300 and the second plane 400, respectively. The display device 120 displays the first projected tool image 310 and the second projected tool image 410 as shadows of the tool cast on the first plane 300 and the second plane 400, respectively, so that the user may better understand the spatial relationship between the object and the tool and may manipulate the tool based on the spatial relationship. The controller 150 may calculate the positions of the first plane 300 and the second plane 400 and the first projected tool image 310 and the second projected tool image 410 of the tool in real time based on image data acquired by the imaging device 110 (e.g., the ultrasound data acquisition device). For example, clips or tags may be attached to the tool so that the position and images of the tool may be generated from the imaging data acquired by the imaging device 110. The projected images of the object and the tool may be edited with different colors. For example, the first projected tool image 310 and/or the first projected object image 320 on the first plane 300 may be different in color from the second projected tool image 410 and/or the second projected object image 420 on the second plane 400, while the projected images on the same plane may have the same color as each other. Alternatively, the first projected tool image 310 and the second projected tool image 410 of the tool may have different color from those of the first projected object image 320 and the second projected object image 420 of the object.

FIG. 7B shows another exemplary view of the object and the tool. As the surgeon pushes the needle further into the skin, the display device 120 may update the 3D tool image 210 of the tool and the first projected tool image 310 and the second projected tool image 410 of the tool as shadows in real time so that the user may manipulate the tool based on the updated spatial relationship between the object and the tool. To further aid the surgeon's manipulation of the tool, the color of the first projected tool image 310 and the second projected tool image 410 of the tool also may be changed to a different color (e.g., red, etc.) once the tool enters the skin. For the same purpose, the lower end position of the tool may be marked with the line 230 (see FIG. 4A) so that the surgeon may easily recognize how far the tool reaches in the skin relative to the object. In this manner, for example, as the user introduces the tool into the skin as shown in FIG. 7A, he/she may be able to recognize from the second projected tool image 410 of the tool is behind the object. As the user pushes the tool further as shown in FIG. 7B, he/she may be able to see that the first projected tool image 310 of the tool reaches the lower edge of the first projected object image 320, and recognize that because the tool reaches the lower edge of the object, he/she does not need to push the tool in further.

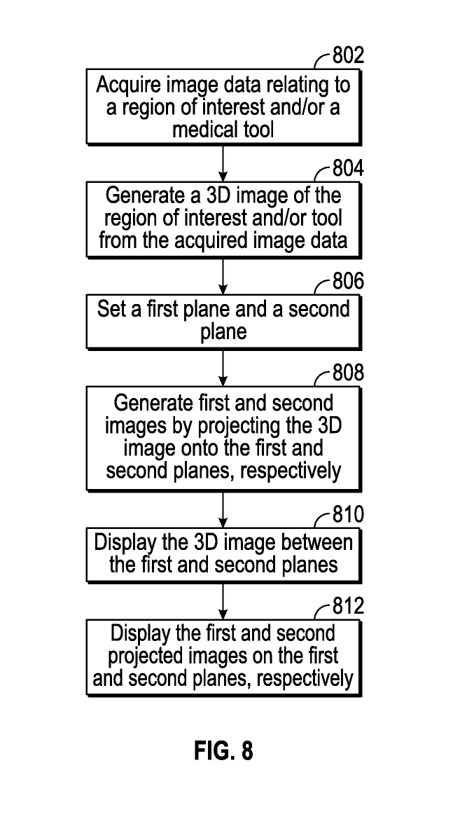

FIG. 8 is a flowchart showing a method for visualizing an object and/or a tool by the visualization system 100, according to an exemplary embodiment.

At step 802, an imaging device (e.g., the imaging device 110, etc.) acquires first image data (e.g., with transducer 122, etc.) relating to an object (e.g., in a human body, a tumor, etc.) in real time. The imaging device may additionally or alternatively acquire second image data relating to a tool (e.g., a needle, scissors, etc.) in real time.

At step 804, the visualization system 100 generates a 3D image of the object (e.g., the 3D object image 220, etc.) based on the first image data. Additionally or alternatively, the visualization system 100 may generate a 3D image of the tool (e.g., the 3D tool image 210, etc.) based on the second image data. Alternatively, the 3D image of the tool may be formed and/or rendered based on a 3D visualization model of the tool stored in memory of the visualization system 100 and positioned relative to the 3D image of the object based on the first image data and/or the second image data.

At step 806, the visualization system 100 sets a first plane (e.g., the first plane 300, a y-z plane, etc.), a second plane intersecting the first plane (e.g., the second plane 400, an x-y plane, perpendicular to the first plane, etc.), and/or a third plane intersecting the first and/or second planes (e.g., an x-z plane, perpendicular to the first and/or second planes, etc.) based on the first image data and/or the second image data.

At step 808, the visualization system 100 generates a first projected image (e.g., the first projected object image 320, etc.), a second projected image (e.g., the second projected object image 420, etc.), and/or a third projected image of the object by projecting a cross-section of the 3D image of the object onto the first plane, the second plane, and/or the third plane, respectively. The visualization system 100 may additionally or alternatively generate a first projected image (e.g., the first projected tool image 310, etc.), a second projected image (e.g., the second projected tool image 410, etc.), and/or a third projected image of the tool by projecting a cross-section of the 3D image of the tool onto the first plane, the second plane, and/or the third plane, respectively.

At step 810, a display device (e.g., the display device 120, etc.) displays the 3D image of the object between the first plane, the second plane, and/or the third plane. The display device may additionally or alternatively display the 3D image of the tool between the first plane, the second plane, and/or the third plane.

At step 812, the display device displays at least one of the first projected image, the second projected image, and/or the third projected image of the object on the first plane, the second plane, and/or the third plane, respectively. The display device may additionally or alternatively display the first projected image, the second projected image, and/or the third projected image of the tool on the first plane, the second plane, and/or the third plane, respectively.

In step 812, the display device may display the projected images of the object and/or the tool with different colors. For example, the projected images of the object and/or the tool on the first plane may be different in color from the projected images of the object and/or tool on the second plane. In some embodiments, the projected images of the object and the tool on the same plane have the same color as each other. Alternatively, the projected images of the tool may have different color from the projected images of the object, while the projected images of the same kind (object or tool) have the same color as each other.

In some embodiments, in step 812, the display device may change the color of the projected images of the tool to a different color (e.g., red, etc.) when the visualization system 100 determines that the tool goes into the skin, e.g., by determining whether a portion of the tool is located inside a surface of a human skin. The display device may also mark the lower end position of the tool with an indicator (e.g., the line 230, etc.) so that the user may easily recognize how far the tool reaches into the skin relative to the object.

As utilized herein, the terms “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the invention as recited in the appended claims.

It should be noted that the term “exemplary” as used herein to describe various embodiments is intended to indicate that such embodiments are possible examples, representations, and/or illustrations of possible embodiments (and such term is not intended to connote that such embodiments are necessarily extraordinary or superlative examples).

The terms “coupled,” “connected,” and the like, as used herein, mean the joining of two members directly or indirectly to one another. Such joining may be stationary (e.g., permanent) or moveable (e.g., removable, releasable, etc.). Such joining may be achieved with the two members or the two members and any additional intermediate members being integrally formed as a single unitary body with one another or with the two members or the two members and any additional intermediate members being attached to one another.

References herein to the positions of elements (e.g., “top,” “bottom,” “above,” “below,” etc.) are merely used to describe the orientation of various elements in the figures. It should be noted that the orientation of various elements may differ according to other exemplary embodiments, and that such variations are intended to be encompassed by the present disclosure.

The present disclosure contemplates methods, systems, and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general purpose computer, special purpose computer, or special purpose processing machines to perform a certain function or group of functions.

It is important to note that the construction and arrangement of the elements of the systems and methods as shown in the exemplary embodiments are illustrative only. Although only a few embodiments of the present disclosure have been described in detail, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts or elements. It should be noted that the elements and/or assemblies of the components described herein may be constructed from any of a wide variety of materials that provide sufficient strength or durability, in any of a wide variety of colors, textures, and combinations. Accordingly, all such modifications are intended to be included within the scope of the present inventions. Other substitutions, modifications, changes, and omissions may be made in the design, operating conditions, and arrangement of the preferred and other exemplary embodiments without departing from scope of the present disclosure or from the spirit of the appended claims.

What is claimed is:

1. A visualization system, comprising:
an imaging device configured to acquire image data relating to an object;
a processing circuit configured to:
generate a three-dimensional object image of the object based on the image data; and
generate a two-dimensional object image of the object by projecting a cross-section of the three-dimensional object image onto a plane; and
a display device configured to:
display the three-dimensional object image of the object; and
display the two-dimensional object image of the object on the plane.

2. The visualization system of claim 1, wherein the two-dimensional object image projected onto the plane includes at least one of a first two-dimensional object image of the object projected onto a first plane, a second two-dimensional object image of the object projected onto a second plane, and a third two-dimensional object image of the object projected onto a third plane.

3. The visualization system of claim 2, wherein:
the imaging device is configured to acquire second image data relating to a tool;
the processing circuit is configured to:
generate a three-dimensional tool image of the tool based on the second image data; and
generate a two-dimensional tool image of the tool by projecting a cross-section of the three-dimensional tool image onto the plane; and
the display device is configured to:
display the three-dimensional tool image of the tool; and
display the two-dimensional tool image of the tool on the plane.

4. The visualization system of claim 3, wherein the two-dimensional tool image projected onto the plane includes at least one of a first two-dimensional tool image of the tool projected onto the first plane, a second two-dimensional tool image of the tool projected onto the second plane, and a third two-dimensional tool image of the tool projected onto the third plane.

5. The visualization system of claim 4, wherein the display device is configured to display at least one of the three-dimensional object image, the first two-dimensional object image, the second two-dimensional object image, and the third two-dimensional object image in a different color than at least one of the three-dimensional tool image, the first two-dimensional tool image, the second two-dimensional tool image, and the third two-dimensional tool image.

6. The visualization system of claim 3, wherein the processing circuit is configured to:
determine whether a portion of the tool is located inside a surface; and
provide a command to the display device to change a color of the two-dimensional tool image to a different color when it is determined that the portion of the tool is located inside the surface.

7. The visualization system of claim 3, wherein the processing circuit is configured to mark a lower end position of the three-dimensional tool image of the tool with a line for display by the display device.

8. The visualization system of claim 1, wherein:
the imaging device is configured to acquire second image data relating to a tool which identifies a presence of the tool and its location;
the processing circuit is configured to:
acquire a three-dimensional model of the tool; and
generate a first two-dimensional tool image of the tool and a second two-dimensional tool image of the tool by projecting a first cross-section of the three-dimensional model onto a first plane and a second cross-section of the three-dimensional model onto a second plane; and
the display device is configured to:
display the three-dimensional model of the tool between the first plane and the second plane; and
display the first two-dimensional tool image and the second two-dimensional tool image on the first plane and the second plane, respectively.

9. The visualization system of claim 1, wherein the processing circuit is configured to:
detect a region of interest based on a preoperative analysis; and
construct a three-dimensional representation of the region of interest including the three-dimensional object image and the two-dimensional object image;
wherein the display device is configured to display the three-dimensional representation of the region of interest.

10. The visualization system of claim 1, wherein the visualization system is an augmented reality based visualization system.

11. The visualization system of claim 1, further comprising an input device configured to facilitate at least one of rotation, translation, and magnification of the three-dimensional object image.

12. A method for visualizing objects using an augmented reality based visualization system comprising:
acquiring, by an imaging device, image data relating to an object;
generating, by a processing circuit, a three-dimensional object image of the object based on the image data;
projecting, by the processing circuit, a two-dimensional object image of the object onto a plane; and
displaying, by a display device, at least one of the three-dimensional object image and the two-dimensional object image.

13. The method of claim 12, wherein projecting the two-dimensional object image onto the plane includes:
projecting, by the processing circuit, a first two-dimensional object image of the object onto a first plane; and
projecting, by the processing circuit, a second two-dimensional object image of the object onto a second plane;
wherein the three-dimensional object image is displayed between the first plane and the second plane.

14. The method of claim 13, further comprising:
acquiring, by the imaging device, second image data relating to a tool;
generating, by the processing circuit, a three-dimensional tool image of the tool based on the second image data;
projecting, by the processing circuit, a first two-dimensional tool image onto the first plane and a second two-dimensional tool image onto the second plane;
displaying, by the display device, the three-dimensional tool image between the first plane and the second plane; and
displaying, by the display device, the first two-dimensional tool image and the second two-dimensional tool image on the first plane and the second plane, respectively.

15. The method of claim 14, wherein at least one of the first two-dimensional object image and the second two-dimensional object image are displayed in a different color than at least one of the first two-dimensional tool image and the second two-dimensional tool image.

16. The method of claim 14, wherein the step of displaying the first two-dimensional tool image and the second two-dimensional tool image includes:
determining, by the processing circuit, whether a portion of the tool is located inside a surface; and
changing, by the processing circuit, a color of at least one of the first two-dimensional tool image and the second two-dimensional tool image to a different color when it is determined that the portion of the tool is located inside the surface.

17. The method of claim 14, wherein the step of displaying the three-dimensional tool image includes marking, by the processing circuit, a lower end position of the three-dimensional tool image with an indicator.

18. The method of claim 12, further comprising steps of:
acquiring, by the imaging device, second image data relating to a tool, wherein the second image data spatially orients the tool in regards to the first image data regarding the object;
acquiring, by the processing circuit, a three-dimensional model of the tool based on the second image data;
projecting, by the processing circuit, a first two-dimensional tool image and a second two-dimensional tool image of the tool onto a first plane and a second plane, respectively;
displaying, by the display device, the three-dimensional model of the tool in relation the three-dimensional object image; and
displaying, by the display device, the first two-dimensional tool image and the second two-dimensional tool image on the first plane and the second plane, respectively.

19. The method of claim 12, further comprising:
detecting, by the imaging device, a region of interest based on preoperative analysis;
constructing, by the processing circuit, a three-dimensional representation of the region of interest including at least one of the three-dimensional object image and the two-dimensional object image; and
displaying, by the display device, the three-dimensional representation of the region of interest.

20. A visualization system, comprising:
a processing circuit configured to:
receive image data from an imaging device regarding an object;
generate a three-dimensional image of the object based on the image data;
set a first plane and a second plane intersecting the first plane;
generate a first two-dimensional image of the object by projecting a first cross-section of the three-dimensional image of the object onto the first plane;
generate a second two-dimensional image of the object by projecting a second cross-section of the three-dimensional image of the object onto the second plane; and
provide a command to a display device to display the three-dimensional image of the object between the first plane and the second plane, the first two-dimensional image of the object on the first plane, and the second two-dimensional image of the object on the second plane.
